(19) United States
(12) Patent Application Publication
(10) Pub. No.: US 2025/0359961 A1
(43) Pub. Date: Nov. 27, 2025

(54) DENTAL POST KIT AND METHOD OF USING THE SAME

(71) Applicant: KING SAUD UNIVERSITY, Riyadh (SA)

(72) Inventors: MOHAMMED ABDULLAH ALSHEHRI, Riyadh (SA); OMAR MOHAMMED ALSHEHRI, Riyadh (SA)

(21) Appl. No.: 18/669,848

(22) Filed: May 21, 2024

(51) Int. Cl. A61C 3/025; A61M 3/02

(52) U.S. Cl. CPC A61C 5/40; A61M 3/0266

(57) ABSTRACT

A dental post kit is provided. The dental post kit includes at least one post having a first section and a second section, where the first section has a first opening, and the second section has a second opening covered by a thin shell. An internal central canal extends from the first opening to the second opening. A stopper is mounted on the at least one post and is located between the first section and the second section. At least one endodontic file is included in the kit. The at least one endodontic file is configured to be inserted into the internal central canal through the first opening and to pass through the second opening by puncturing the thin shell covering.

### BACKGROUND

### Field

The disclosure of the present application relates to dentistry, and particularly to a dental post kit and a method for using the dental post kit for an endodontic treatment.

### Description of Related Art

A human tooth includes an enamel layer which is a hard outermost coating of the tooth. The enamel forms a crown of the tooth. Beneath the enamel is dentine, which is a softer material that supports the enamel and forms most of the tooth. The dentine has a plurality of small tubes that connect directly with the tooth's pulp chamber. The pulp chamber is located in the middle of the tooth and extends from the crown of the tooth into the root of the tooth. The root, which contains at least one root canal, is the part of the tooth that extends into the bone and holds the tooth in place. The pulp chamber contains pulp, which is made of nerves, blood vessels, and lymphatics. The pulp is located within the at least one root canal in each root. The root's surface is coated by a hard material called cementum.

Depending on the tooth, the number of roots and root canals can vary. For example, incisors and canines usually have only one root and one root canal. Premolars can have one or two roots with one or two corresponding root canals. Molars can have two or three roots with three or more corresponding root canals.

Bacteria, which can enter through either a dental cavity or a chip or crack in the tooth, can infect the pulp, causing it to become inflamed. If left untreated, the bacteria can multiply and spread throughout the pulp and the root canal system. Signs and symptoms of a root canal infection can include severe tooth pain when biting or chewing, swollen and tender gums, pus around an infected tooth, sensitivity to hot or cold food or liquids, a loose tooth, swelling of the neck of face, and darker coloration of a tooth.

To relieve the pain or discomfort caused by the infection, the pulp is removed via an endodontic treatment or root canal treatment. During cleaning and shaping of the at least one root canal using an endodontic file, small fragments of debris accumulate on the walls of the at least one root canal, forming a smear layer. The smear layer contains at least fragments of odontoblastic processes, bacteria, and necrotic debris. Removal of the smear layer during the endodontic treatment is important for cleanliness and disinfection of the at least one root canal, resulting in a better adaptation of root canal filling material. Additionally, removal of the smear layer also prevents reinfection of the at least one root canal.

The use of the endodontic file during the cleaning and shaping steps poses several problems. First, insertion of the endodontic file within the at least one root canal is usually performed by the guidance of tactile sensation of the endodontist. This method may result in the endodontic file moving in the wrong direction, thus unintentionally removing a portion of the dentin. Second, misguidance of the endodontic file may also result in perforation of the at least root canal. This can cause periodontal tissue damage and esthetic problems. Thus, a dental post kit and a method of using the same solving the aforementioned problems are desired.

### SUMMARY

The present disclosure relates to a dental post kit which, in an embodiment, includes at least one post having a first section and a second section, where the first section has a first opening, and the second section has a second opening with a thin shell covering the second opening. An internal central canal extends from the first opening to the second opening. A stopper may also be provided as part of the kit, where the stopper is located between the first section and the second section of the at least one post. The kit also includes at least one endodontic file configured to be inserted into the internal central canal through the first opening and passing through the second opening by puncturing the thin shell covering.

As a non-limiting example, the first section can include a coronal part with a coronal width of about 1.3 mm to about 1.9 mm. As a further non-limiting example, the second section can include an apical tip with an apical width of about 0.7 mm to about 1.0 mm. As a further non-limiting example, a diameter of the internal central canal can be about 1/10 to about ⅓ of a diameter of the at least one post.

As a non-limiting example, the at least one post can be a mixture of fiber and resin. As a further non-limiting example, the fiber may be carbon, glass, silica, quartz, other fibers, or combinations thereof, and the resin may be epoxy, bisphenol A-glycidyl methacrylate (bis-GMA), other resins, or combinations thereof.

As non-limiting examples, the internal central canal can be a straight opening or a tapered opening. As a further non-limiting example, the stopper can be a removable, slidable rubber stopper which can be configured to slide on and off the at least one post. As another non-limiting example, the at least one endodontic file can have a length of about 16 mm.

The at least one endodontic file can include a first end and a second end and, as a non-limiting example, the second end can have a fixed taper of about 7%.

In use, an opening is drilled into a crown and a pulp chamber of a tooth of an individual experiencing tooth pain or sensitivity in order to access at least one root canal of the tooth. At least one intracanal irrigant is injected into the at least one root canal of the tooth, and the at least one post is inserted into the at least one root canal of the tooth. The at least one endodontic file is inserted into the first opening of the at least one post and then pushed toward the second opening to puncture the thin shell of the at least one post, thus allowing the at least one endodontic file to enter the at least one root canal of the tooth. The at least one endodontic file is moved within the at least one root canal to clean and shape the at least one root canal.

As a non-limiting example, the at least one intracanal irrigant may be sodium hyphochlorite (NaOCl), chlorhexidine, ethylenediaminetetraacetic acid (EDTA), tetracycline, MTAD™, other intracanal irrigants, and combinations thereof. MTAD™ is a mixture of doxycycline, citric acid, and a detergent (Tween 80), and is sold by Dentsply® International Inc. of Delaware. As a further non-limiting example, the step of cleaning the at least one root canal can include moving the at least one endodontic file within the at least one root canal to cause the at least one intracanal irrigant to be agitated, thus removing a smear layer within the at least one root canal.

Following the above procedure, the at least one post is withdrawn, with the at least one endodontic file attached thereto, from the at least one root canal and the tooth. The at least one endodontic file is removed from the at least one post, and the cleaned and shaped at least one root canal is filled with a filler material. The at least one post is then reinserted into the at least one root canal, attaching a portion of the at least one post to the filler material. An adhesive cement is injected into the opening of the crown, the opening of the pulp chamber, and the at least one root canal, thus sealing the crown, the pulp chamber, and the at least one root canal. A replacement crown may then be placed on the tooth. As a non-limiting example, the filler material can be gutta-percha. As a further non-limiting example, the gutta-percha can be heated and compacted prior to the step of reinserting the at least one post into the at least one root canal.

These and other features of the present subject matter will become readily apparent upon further review of the following specification.

### BRIEF DESCRIPTION OF DRAWINGS

FIG. 1 is a perspective view of a dental post of the dental post kit.

FIG. 2 is a side view in section of the dental post of FIG. 1.

FIG. 3 is a side view in section of the dental post of FIG. 1 with an attached endodontic file, provided as part of the dental post kit.

Similar reference characters denote corresponding features consistently throughout the attached drawings.

### DETAILED DESCRIPTION

The following definitions are provided for the purpose of understanding the present subject matter and for construing the appended patent claims. The definitions are not meant to be limiting to the subject matter described herein.

### Definitions

Throughout the application, where systems are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a system or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

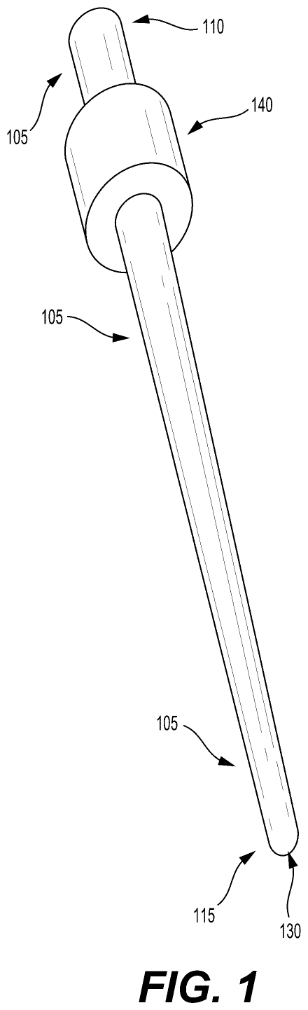

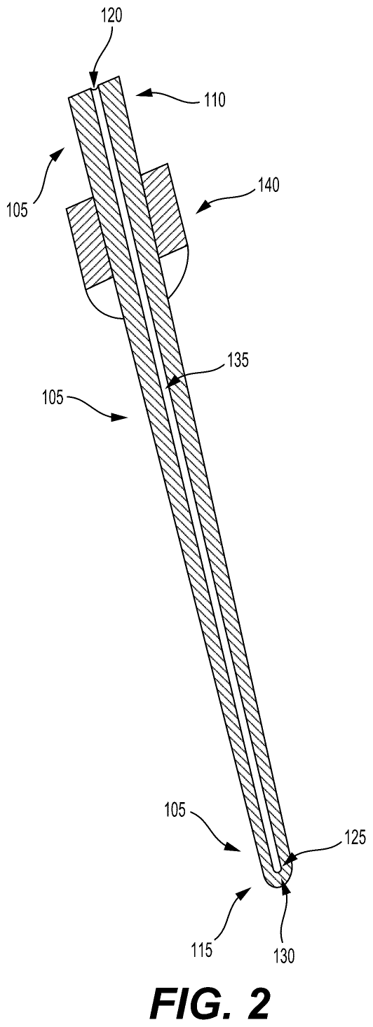

FIGS. 1-3 depict, in an embodiment, a dental post (105) having a first section (110) and a second section (115). It should be understood that the dental post (105) may be provided as part of a kit including at least one such dental post (105). In a non-limiting embodiment, the at least one post (105) can be flexible and inelastic and can be formed from a mixture of fiber and resin. As a non-limiting example, the fiber may be carbon, glass, silica, quartz, other fibers, or combinations thereof. As a non-limiting example, the resin may be epoxy, bisphenol A-glycidyl methacrylate (bis-GMA), other resins, or combinations thereof. As shown in FIG. 2, the first section (110) has a first opening (120) formed therein. The second section (115) has a second opening (125) which, as shown in FIG. 2, is covered by a thin shell (130).

The first section (110) can include a coronal part with a coronal width. As a non-limiting example, the coronal width can be about 1.3 mm to about 1.9 mm. The second section (115) can include an apical tip with an apical width. As a non-limiting example, the apical width can be about 0.7 mm to about 1.0 mm. An internal central canal (135) is shown, which extends from the first opening (120) to the second opening (125). The internal central canal (135) can be a straight passage or a tapered passage. It has been found that straight passages of the internal central canal (135) are stronger than tapered passages. As a non-limiting example, the diameter of the internal central canal (135) can be about 1/10 to about ⅓ of the diameter of the at least one post (105).

A stopper (140) is shown, which is located between the first section (110) and the second section (115) of the at least one post (105). The stopper (140) may be provided as part of the kit. The stopper (140) can be a removable, slidable rubber stopper, for example, which can be configured to slide on and off the at least one post (105). By way of a non-limiting example, the stopper (140) can slide onto the at least one post (105) by pushing the second section (115) of the at least one post (105) through an orifice (not shown) of the stopper (140), thereby allowing the stopper (140) to be located between the located between the first section (110) and the second section (115) of the at least one post (105).

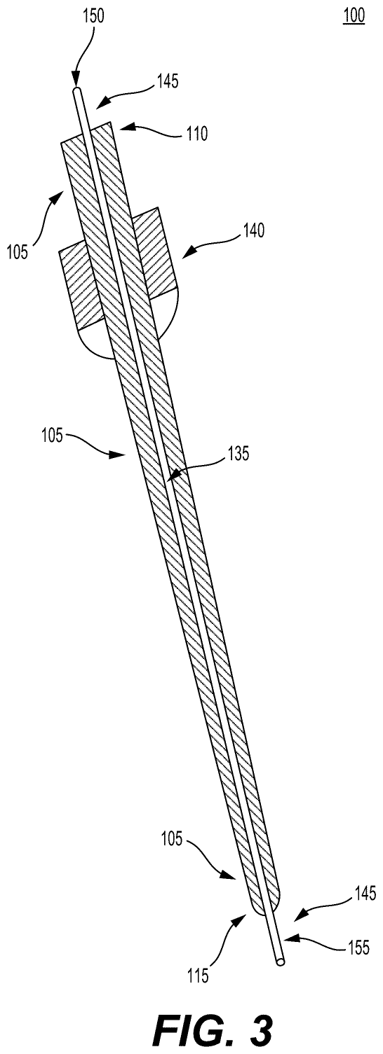

As shown in FIG. 3, at least one endodontic file (145) may be provided as part of the kit. The at least one endodontic file (145) may be configured to be inserted into the internal central canal (135) through the first opening (120) and passing through the second opening (125) by puncturing the thin shell (130). The at least one endodontic file (145) includes a first end (150) and a second end (155). In FIG. 3, the second end (155) is shown as having punctured the thin shell 130, thus passing through out and out of second opening (125). As a non-limiting example, the second end (155) can have a fixed taper of about 7%. As another non-limiting example, the at least one endodontic file (145) can have a length of about 16 mm. As further non-limiting examples, the at least one endodontic file (145) can be a size 06 file, a size 08 file, a size 10 file, or a size 15 file with a tip thickness of about 0.06 mm, 0.08 mm, 0.10 mm, and 0.15 mm, respectively.

In use, an opening is drilled into a crown and a pulp chamber of a tooth of an individual experiencing tooth pain or sensitivity to access at least one root canal of the tooth. At least one intracanal irrigant is injected into the at least one root canal of the tooth, and the at least one post (105) is inserted into the at least one root canal of the tooth. The at least one endodontic file (145) is inserted into the first opening (120) of the at least one post (105), and the at least one endodontic file (145) is pushed toward the second opening (125) to puncture the thin shell (130) of the at least one post (105), allowing the at least one endodontic file (145) to enter the at least one root canal of the tooth. The at least one endodontic file (145) is moved within the at least one root canal to clean and shape the at least one root canal. Prior to the puncturing step, and during the insertion of the at least one post (105) into the at least one root canal of the tooth, the thin shell (130) of the at least one post (105) prevents any preexisting adhesive cement within the tooth from entering the internal central canal (135), thus facilitating the insertion of the at least one endodontic file (145) into the internal central canal (135). Additionally, by restricting the movement of the at least one endodontic file (145) within the internal central canal (135) of the at least one post (105), the problems mentioned previously during the cleaning and shaping steps of the at least one root canal are alleviated.

As a non-limiting example, the at least one intracanal irrigant may be sodium hyphochlorite (NaOCl), chlorhexidine, ethylenediaminetetraacetic acid (EDTA), tetracycline, MTAD™, other intracanal irrigants, and combinations thereof. MTAD™ is a mixture of doxycycline, citric acid, and a detergent (Tween 80), and is sold by Dentsply® International Inc. of Delaware. As a further non-limiting example, the step of cleaning the at least one root canal can include moving the at least one endodontic file within the at least one root canal to cause the at least one intracanal irrigant to be agitated thereby removing a smear layer within the at least one root canal.

Following the above procedure, the at least one post (105), with the at least one endodontic file (145) attached thereto, is withdrawn from the at least one root canal and the tooth (after the steps of cleaning and shaping the at least one root canal). The at least one endodontic file (145) is removed from the at least one post (105), and the cleaned and shaped at least one root canal is filled with a filler material. The at least one post (105) is reinserted into the at least one root canal, attaching a portion of the at least one post to the filler material. An adhesive cement is injected into the opening of the crown, the opening of the pulp chamber, and the at least one root canal, thus sealing the crown, the pulp chamber, and the at least one root canal. A replacement crown is then placed on the tooth.

As a non-limiting example, the filler material can be gutta-percha. As a further non-limiting example, the gutta-percha can be heated and compacted prior to the step of reinserting the at least one post (105) into the at least one root canal.

It is to be understood that the dental post kit and the method of using the same are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A dental post kit, comprising:
at least one post comprising a first section and a second section, the first section having a first end terminating with a first opening formed therein, the second section having a second end therein, wherein the second end is covered by a shell, wherein an internal central canal extends from the first opening to the second end, and wherein the at least one post is configured to be inserted into at least one root canal of a tooth of a patient;
a stopper mounted on the at least one post, the stopper being located between the first section and the second section of the at least one post, wherein the stopper is removable and slidably mounted on the at least one post such that the stopper is slidable along said first section of said at least one post and pushes said second section of said at least one post; and
at least one endodontic file configured to be inserted into the internal central canal through the first opening and pass through the second end by puncturing the shell, thereby forming a second opening for the second section.

2. The dental post kit of claim 1, wherein the first section comprises a coronal part with a coronal width of about 1.3 mm to about 1.9 mm.

3. The dental post kit of claim 1, wherein the second section comprises an apical tip with an apical width of about 0.7 mm to about 1.0 mm.

4. The dental post kit of claim 1, wherein a diameter of the internal central canal is about 1/10 to about ⅓ of a diameter of the at least one post.

5. The dental post kit of claim 1, wherein the at least one post comprises a mixture of fiber and resin.

6. The dental post kit of claim 5, wherein the fiber is selected from the group consisting of carbon, glass, silica, quartz, and combinations thereof.

7. The dental post kit of claim 5, wherein the resin is selected from the group consisting of epoxy, bisphenol A-glycidyl methacrylate (bis-GMA), and combinations thereof.

8. The dental post kit of claim 1, wherein the internal central canal is a straight passage.

9. The dental post kit of claim 1, wherein the internal central canal is a tapered passage.

10. The dental post kit of claim 1, wherein the stopper is a removably and slidably mounted on the at least one post.

11. The dental post kit of claim 1, wherein the at least one endodontic file has a length of about 16 mm.

12. The dental post kit of claim 1, wherein the at least one endodontic file comprises a first end and a second end, wherein the second end has a fixed taper of about 7%.

13. A method of performing a root canal procedure, comprising:
drilling an opening into a crown and a pulp chamber of a tooth of patient to access at least one root canal of the tooth;
injecting at least one intracanal irrigant into the at least one root canal of the tooth;
providing at least one post comprising a first section and a second section, the first section having a first opening formed therein, the second section having a second opening formed therein, wherein the second opening is covered by a shell, wherein an internal central canal extends from the first opening to the second opening, and wherein a stopper is mounted on the at least one post, the stopper being located between the first section and the second section of the at least one post, wherein the stopper is removable and slidably mounted on the at least one post such that the stopper is slidable along said first section of said at least one post and pushes said second section of said at least one post;
inserting the at least one post into the at least one root canal of the tooth;
inserting at least one endodontic file into a first opening of the at least one post;
pushing the at least one endodontic file toward the second opening to puncture the shell of the at least one post such that the at least one endodontic file enters the at least one root canal of the tooth; and
moving the at least one endodontic file within the at least one root canal to clean and shape the at least one root canal.

14. The method of claim 13, wherein the at least one intracanal irrigant is selected from the group consisting of sodium hyphochlorite (NaOCl), chlorhexidine, ethylenediaminetetraacetic acid (EDTA), tetracycline, MTAD™, and combinations thereof.

15. The method of claim 13, wherein the step of cleaning the at least one root canal comprises moving the at least one endodontic file within the at least one root canal to cause the at least one intracanal irrigant to be agitated such that a smear layer is removed within the at least one root canal.

16. The method of claim 13, further comprising:
withdrawing the at least one post with the at least one endodontic file attached thereto from the at least one root canal and the tooth following the steps of cleaning and shaping the at least one root canal;
removing the at least one endodontic file from the at least one post;
filling the cleaned and shaped at least one root canal with a filler material;
reinserting the at least one post into the at least one root canal to attach a portion of the at least one post to the filler material;
injecting an adhesive cement into the opening of the crown, the opening of the pulp chamber, and the at least one root canal to seal the crown, the pulp chamber, and the at least one root canal; and
placing a replacement crown on the tooth.

17. The method of claim 16, wherein the filler material comprises gutta-percha.

18. The method of claim 17, wherein the gutta-percha is heated and compacted prior to the step of reinserting the at least one post into the at least one root canal.

19. The method of claim 13, wherein the internal central canal comprises a straight passage or a tapered passage.

20. The method of claim 13, wherein the stopper is a removably and slidably mounted on the at least one post.
